(19) United States
(12) Patent Application Publication
(10) Pub. No.: US 2026/0069775 A1
(43) Pub. Date: Mar. 12, 2026

(54) INFUSION VOLUME DETECTION SYSTEM AND METHOD

(71) Applicant: CareFusion 303, Inc., San Diego, CA (US)

(72) Inventors: Ronald HIDALGO, Pallaskenry (IE); Suman HARAPANAHALLI, Tralee (IE); Ayesha JAVAID, Kildare (IE); Diarmuid KEARNEY, Co Limerick (IE); Geanderson TEIXEIRA, Tulla (IE)

(21) Appl. No.: 18/827,476

(22) Filed: Sep. 6, 2024

(51) Int. Cl. A61M 5/168; A61M 5/172

(52) U.S. Cl. CPC A61M 5/16886; A61M 5/1723

(57) ABSTRACT

A vision-enabled fluid flow rate detection system is disclosed. The system includes an image sensing device that images an infusion container supplying a fluid to an infusion pump and a pattern of markings associated with a surface of the container, and identifies a visual difference in the pattern from a default state of the pattern to determine a volume of fluid infused from the infusion container, and used to calculate a volume of fluid in the container and a flow rate of the fluid. When the amount of fluid, as determined by the vision system, differs from the amount reported by the pump, the pump's motor may be adjusted to correct the expected volume or flow rate, as needed. When a severe infusion inaccuracy is detected, an alarm or other indication may be provided, or the infusion terminated.

### BACKGROUND

In medical care facilities, infusion of medical fluids into a patient is a commonly performed patient care operation. A fluid infusion device, such as an infusion pump, is typically configured to infuse a fluid from a fluid source into a patient through a vascular access device (VAD) such as a syringe or a catheter.

Over-infusion and under-infusion of medication while using an infusion pump are issues that can happen in the patient setting. Modern infusion devices include built-in sensors for detecting when a predetermined volume is infused and, while there are systems in place to generate alarms based on alarm thresholds for fluid flow, the pump itself may report an incorrect flow rate or volume infused. There is a number of factors that cause these inaccuracies including, but not limited to: (1) pump-to-pump variation due to tolerance stack up of the many individual components; (2) environmental setting, e.g. temperature, relative humidity; (3) medication fluid properties, e.g. viscosity, density; (4) fluid bag height; (5) variations in the disposable sets. While a clinician may be aware of these factors when initiating an infusion therapy, the factors may not always be taken into account in a real clinical setting. For example, the fluid bag may not be set at the recommended height. Inaccuracies in the pump's encoder may further contribute to inaccurate measurements and reporting provided by the pump.

### SUMMARY

The subject technology provides a vision-enabled system that monitors fluid levels in a fluid container used in an infusion therapy, compares the monitored fluid levels to fluid-related parameters reported by conventional systems, and adjusts the infusion therapy as needed during runtime to ensure accuracy of the infusion therapy. The adjustment may be, for example, provided as closed-loop feedback that makes adjustments to parameters of an infusion pump, including adjusting a motor speed of the pump to correct expected infused volume at a given time.

According to various aspects of the subject technology, the disclosed fluid flow detection system comprises: an image sensing device configured to image an infusion container supplying a fluid to an infusion pump; a pattern of markings associated with a surface of an infusion container and positioned such that the pattern is superimposed with a characteristic of a fluid within the infusion container when the infusion container is imaged by the image sensing device; and one or more processors configured to: cause the image sensing device to image the pattern of markings during an infusion of the fluid by the infusion pump; determine, based on the imaging of the pattern, a visual difference in the pattern from a default state of the pattern; determine a volume of fluid infused from the infusion container based on the visual difference in the pattern; determine a flow rate of infusion; and provide an indication of the volume of fluid infused from the infusion container on a display device associated with the infusion pump. Other aspects include corresponding methods, apparatus, and computer program products for implementation of the corresponding system and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example pump-driven fluid delivery system, including an infusion pump shown in use in its intended environment, according to various aspects of the subject technology.

FIGS. 2A and 2B depict an example pattern of markings associated with a surface of an infusion container, according to various aspects of the subject technology.

FIG. 3A-3C depict an example vision-enabled fluid-flow detection system, according to various aspects of the subject technology.

FIG. 4 depicts a first example process for maintaining accuracy of an infusion pump using a vision-enabled fluid-flow detection system, according to various aspects of the subject technology.

FIG. 5 depicts a second example process for maintaining accuracy of an infusion pump using a vision-enabled fluid-flow detection system, according to various aspects of the subject technology.

FIG. 6 depicts an example process for training the disclosed vision-enabled fluid-flow detection system to detect on or more patterns associated with a fluid container to detect fluid flow during a patient therapy, according to various aspects of the subject technology.

FIG. 7 is a conceptual diagram illustrating an example electronic system for maintaining accuracy of an infusion pump using a vision-enabled fluid-flow detection system, according to aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The disclosed fluid flow detection system augments a volumetric infusion pump with a connected vision system that includes a camera module. The vision system communicates with the pump and adds a means of checking the actual amount of volume infused by measuring the fluid level reduction in the infusion fluid container (e.g. the medication bag), for example, by characterizing a fluid level reduction rate according to the bag size. The means may include a statistical model or a machine learning model that is trained to estimate volume based on an image captured by a vision system. The continuous fluid level reduction rate feedback from the vision system gives the volumetric pump the capability to adjust parameters such as the pump's motor speed to deliver the correct expected infused volume for a given time. The disclosed system is further capable of checking for severe infusion inaccuracies and can provide early detection, alarm, or termination of the infusion, as necessary.

Providing a real-time, infused volume feedback (via pre-determined fluid levels) from the disclosed vision system has several advantages. For example, if there is a discrepancy between actual infused volume and the expected volume infused at a given time, the pump can adjust its parameters to bring the actual infused volume to match the expected volume. One parameter can be the motor speed: if there is over-infusion, the pump may decrease motor speed; if under-infusion, pump may increase motor speed, thereby minimizing delivery inaccuracies. At severe conditions of inaccuracies, which may be defined by the manufacturer (for example, 40% under-infusion) the extreme difference between the actual infused volume and the expected volume can trigger an alarm or can stop the infusion, depending on the workflow as defined by the manufacturer. This ability to detect such severe inaccuracies in real-time can avoid potential harm to the patient as early as possible.

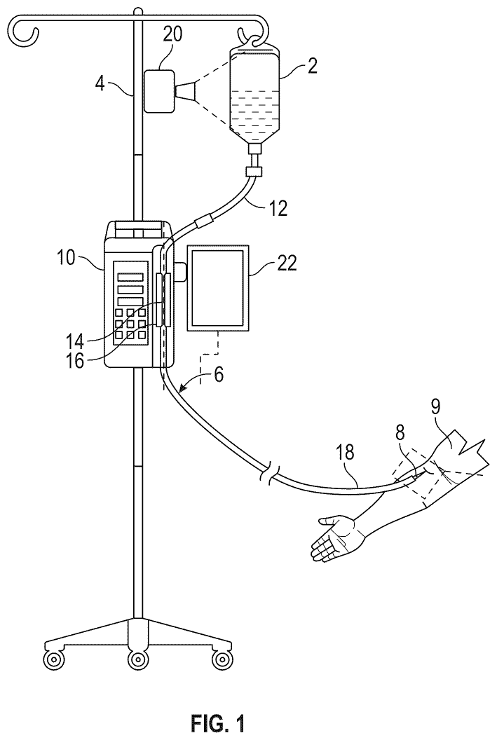

FIG. 1 depicts an example pump-driven fluid delivery system, including an infusion pump 10 shown in use in its intended environment, according to various aspects of the subject technology. In the depicted example, a fluid source 2 containing an intravenous (IV) fluid is held on an intravenous (IV) pole 4. According to various implementations, the fluid source is a malleable fluid container such as an IV bag or blood product bag. An infusion line 6 is connected to the malleable fluid container 2 for delivery of the fluid to the patient. The infusion line 6 may be a conventional IV infusion-type tube typically used in a hospital or medical environment, and is made of any type of flexible tubing appropriate for use to infuse therapeutic fluids into a patient, such as polyvinylchloride (PVC). A cannula 8 is mounted at the distal end of the flexible IV tubing for insertion into a patient's blood vessel or other body location 9 to deliver the fluid to the patient.

The fluid source is connected in fluid communication with an upstream portion 12 of the fluid line 6. A flexible portion 14 of the fluid line is mounted in operative engagement with a peristaltic pumping apparatus 16 for propelling fluid through a downstream portion 18 of the fluid line 6, for example, to a patient's arm 9.

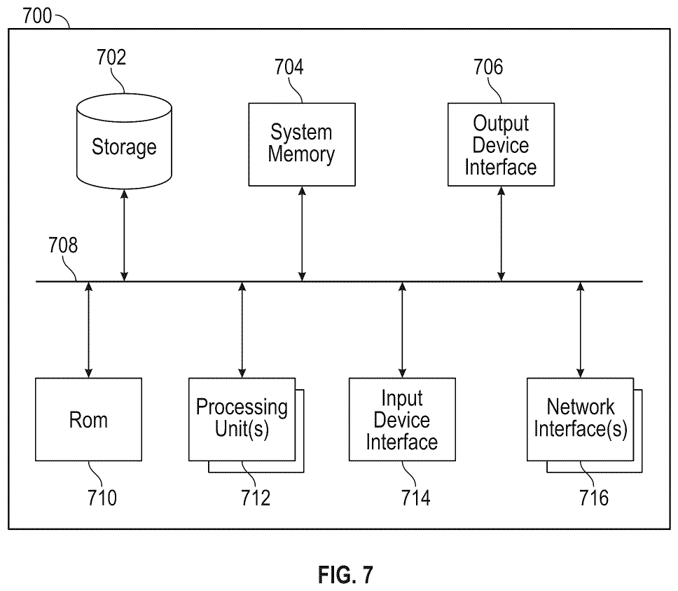

In some implementations, a pump controller may control the flow rate of fluid through the IV tube by way of modulating the speed of the pump. The pump controller may be implemented by or include one or more processors or an electronic system for controlling operations of the infusion pump (see FIG. 7). In this regard, the pump controller modulates the flow of the fluid in an ongoing infusion by way of controlling a pumping mechanism of the pump (e.g., by way of electrical signals and/or software operations). The controller may also interface with the tube (e.g., downstream of the pump) by way of controlling a pinching device that pinches the tube more or less to control the flow of fluid therethrough. The controller's modulation of the fluid may be responsive to a control signal generated by, for example, a flow sensor attached to the drip chamber, or feedback signals from the vision system described herein.

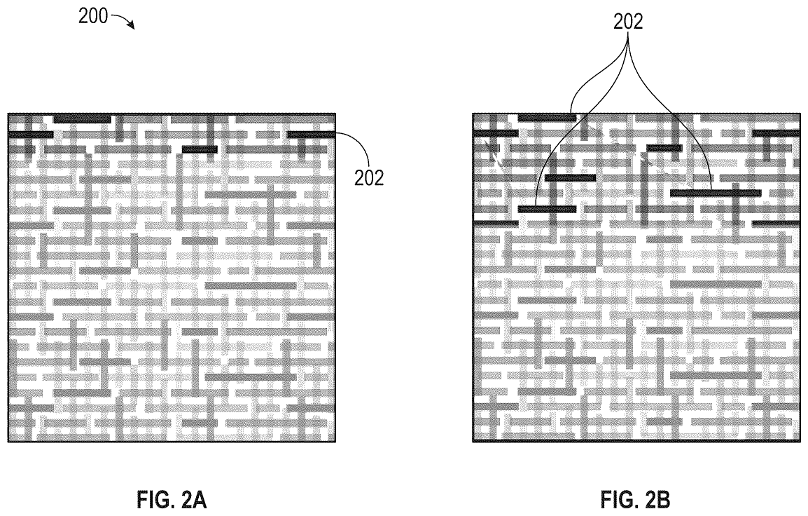

As will be described further, an image sensing device 20 may be attached to the IV pole 4 to image the fluid container 2 and to determine an amount of remaining fluid within the container. In some implementations, a computing device 22 and/or display, may also be attached to the IV pole to facilitate readings from the image sensing device 20. For example, as shown in FIG. 2, an image sensing device may be configured to the fluid level from the container and transmit the information to the computing device 22 for interpretation by a user.

FIGS. 2A and 2B depict an example pattern of markings 200 associated with a surface of an infusion container 2, according to various aspects of the subject technology. According to various implementations, a pattern of markings 200 is printed or otherwise affixed to a translucent fluid container 2. The pattern of markings may include, for example, a plurality of straight lines or symbols. The lines may be semi-transparent and/or may include color or hue properties that, when illuminated, are modified by a fluid behind the pattern within the container. In some implementations, the lines may further deform (e.g., take on a curvature) as fluid is depleted from the container 2. Accordingly, the pattern becomes superimposed with a characteristic of a fluid within the infusion container when the infusion container is imaged by the image sensing device 20.

In some implementations, a fluid container may be associated with a background pattern and a foreground pattern. In some implementations, the background pattern may include the side of the medication container (e.g., side of the bag) furthest from the image sensing device 20 being a uniform color or pattern. For example, the background pattern may be a solid color that provides maximum contrast with the fluid.

Accordingly, when the infusion container is imaged by the image sensing device, the foreground pattern is in front of the fluid within the infusion container, between the image sensing device 20 and the infusion container 2, and the background pattern is behind the fluid in the infusion container 2. Because the infusion container 2 is transparent, the image generated by the image sensing device 20 includes the foreground pattern being visually modified by a combination of the background pattern and a characteristic of the fluid (e.g., color, reflective property, hue, and the like) within the infusion container. Moreover, portions of the modified foreground pattern may be different based on a presence or absence of the fluid within the infusion container at the portions.

For example, FIG. 2A depicts a fluid level at a higher level than in FIG. 2B. As the fluid empties from the container, the lines may become darker 202, indicating empty space within the container 2. In some implementations, a volume infused from the container 2 can be estimated using a difference between two images; e.g., between FIGS. 2A and 2B. In the depicted examples, the fluid line in the pattern of FIG. 2A is compared to fluid line in the image of FIG. 2B to assess the volume infused. In such implementations, the algorithm does not necessarily need to look for the fluid line but rather can look at shapes of a non-repeating pattern or, when color is used, color histogram differences to gauge the amount of fluid expelled between time 1 and time 2.

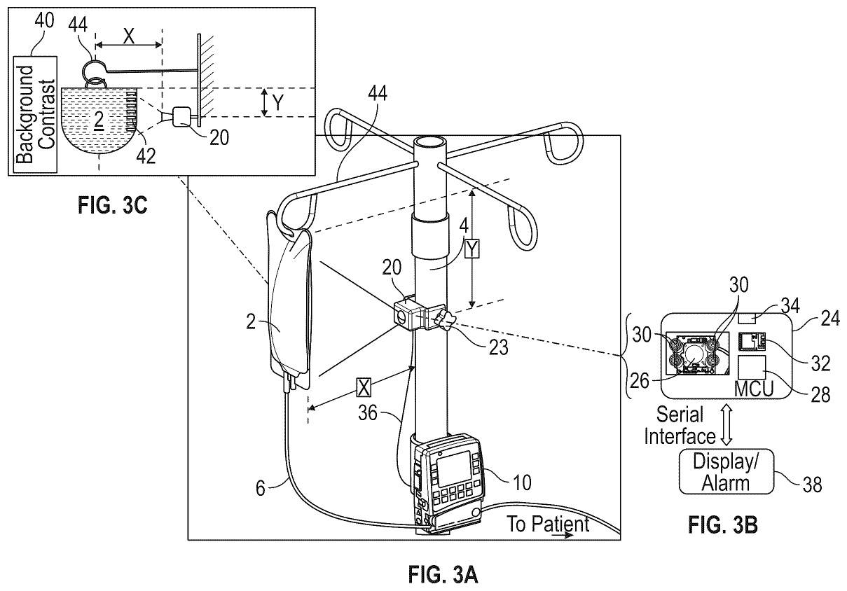

FIG. 3A-3C depict an example vision-enabled fluid-flow detection system, according to various aspects of the subject technology. The depicted system includes an image sensing device 20 such as a camera capable of capturing images for subsequent vision processing by a processor. As will be described further, the image sensing device 20 is located and positioned so that the camera is aligned with a pattern of markings printed or otherwise affixed to the fluid container 2. The fluid-flow detection system includes computer vision software configured to operate the image sensing device 20 to recognize objects and interpret the patterns of FIGS. 2A and 2B. Collectively, the software and camera and related hardware may be termed herein as the disclosed “vision system. ”

As shown in FIG. 3A, the fluid container 2 (e.g., not necessarily a soft bag) is hung on a fixture; for example, the same fixture that mounts the image sensing device 20. In some implementations, the image sensing device 20 includes a coupling mechanism 23 that couples the sensing device 20 to the pole 4 and aligns the image sensing device 20 with the fluid container 2, as shown. In the depicted example, the coupling mechanism 23 is adjustable to move the sensing device 20 up or down on the pole 4, vertically.

As shown in FIG. 3B, the image sensing device 20 may include or be connected to one or more processors. In the depicted example, device 20 includes an internal circuit board 24 with a miniature camera 26 and a microprocessor 28 operating the camera 26 for sensing a pattern of markings associated with the fluid container 2. An LED array 30 may be included to illuminate the markings for the camera 26. The internal circuit board 24 may further include a wireless circuit 32 (e.g., Bluetooth or RF communication circuit), or a wired interface 34 (e.g., a USB communication interface), for communication over a wired connection 36 with a remote computer system or an operably coupled infusion device 10. In some implementations, a serial interface 38 may be included for interfacing with external devices, such as an external display or alarm. In some implementations, an external computing device (e.g., a mobile device) may operably connect to the device 20 via the wireless interface or wired interface and control operation of, or collect data from, the device. For example, the sensing device 20 and/or an operable connected server connected to the device 20 may notify a clinician about a detected fluid level via the clinician's mobile phone or device (via Bluetooth or Internet connection). In some implementations, various operations of an infusion device 10 may be triggered by signals provided by the device 20 responsive to sensing a threshold fluid level drop in the fluid container 2.

According to various implementations, the processor 28 of the device 20 (or, e.g., a remotely connected processor) may be programmed to cause the image sensing device 20 to read the marking(s) on a surface of the fluid container 2, and then based on the markings, measure a current variation in the markings from a default state. As described previously, the markings may include a plurality of straight lines when the bag is full of fluid. In some implementations, when the fluid container is deformable (e.g., an IV bag), the lines may deform into a pattern of curved lines according to an amount of curvature as the fluid is depleted from the container. The processor is programmed to detect and match the pattern of lines with one or more predetermined patterns, and determine the current volume of the fluid within the container based on indexing a matched pattern with a predetermined fluid level. In this regard, the processor(s) may be further programmed to determine an expansion state of a deformable medication container—e.g., a fluid bag—based on the markings read from the surface of a side of the container. For example, the expansion state may include an amount of shape change in the pattern (and, in some implementations, a curvature change in the lines of the pattern) from a default state. The processor(s) may be further programmed to determine a deviation in an initial fluid level and/or volume within the container from a baseline fluid level and/or volume based on the change in the pattern.

With brief reference to FIG. 3C, the fluid container 2 may include a rigid and transparent body to maintain a consistent shape for a more accurate fluid level detection and lined with a fluid level scale for the image sensing device 20 to refer to for fluid level detection. In all or some implementations, a background surface 40 may be provided for the container 2 that provides maximum contrast with the fluid within the container 2. Additionally, the foreground pattern 42 may include distinguishable lines for detection (with the background and fluid) of the fluid level within the fluid container 2, as shown. In some implementations,

As shown, a bespoke hook design 44 may be implemented to keep the fluid container 2 stable during the therapy. The mounting location of the fluid container 2 may be consistent, for example, by specifying the X and Y distances of a datum feature of the fluid container 2 relative to a datum feature in the camera module of the image sensing device 20.

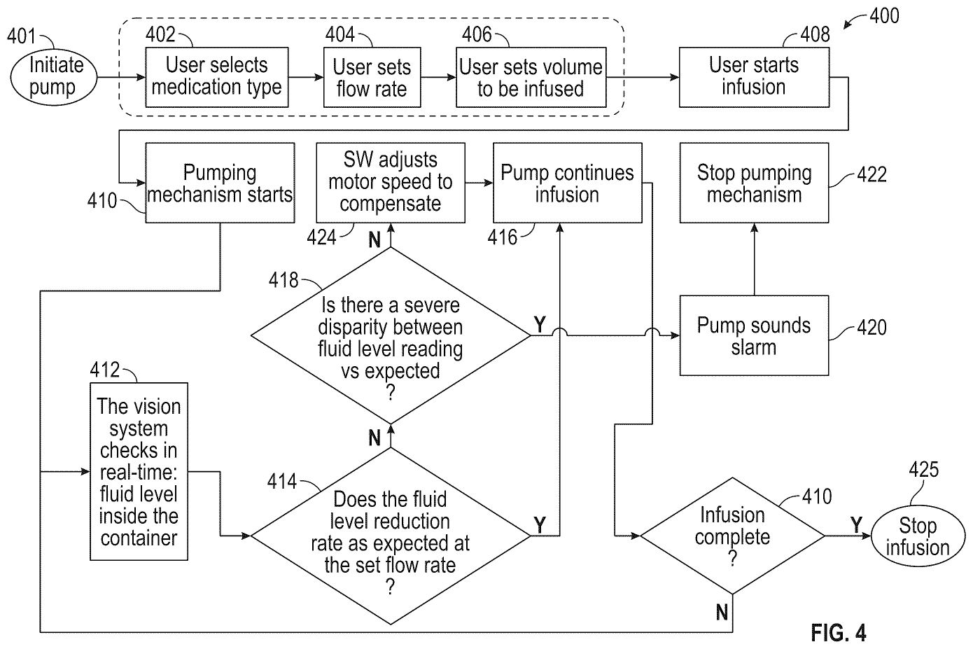

FIG. 4 depicts a first example process 400 for maintaining accuracy of an infusion pump using a vision-enabled fluid-flow detection system, according to various aspects of the subject technology. For explanatory purposes, the various blocks of example process 400 are described herein with reference to FIGS. 1-3, as well as the components and processes described herein. In some implementations, one or more of the blocks may be implemented apart from other blocks, and by one or more different processors or devices. Further, for explanatory purposes, the blocks of example process 400 are described as occurring in serial, or linearly. However, multiple blocks of example process 400 may occur in parallel. In addition, the blocks of example process 400 need not be performed in the order shown and one or more of the blocks of example process 400 need not be performed.

The addition of the disclosed image sensing device 20 adds a layer of checks during an infusion to make sure that the actual amount of infused volume of medication matches what is expected. In the depicted example, an infusion pump 10 is initiated (401). In this regard, the pump 10 receives data of characterized fluid levels expected per flow rate (e.g., stored via an internal memory or external memory attached to it). The characterization of the fluid levels may include the various medication container sizes that the infusion pump supports. The user selects the medication type (402), sets the flow rate (404) and the volume to be infused (VTBI) (406), and then starts the infusion (408). In some implementations, these input steps may be auto-populated by way of an automated programming request (APR) sent to the pump by a server or other computing device responsive to a scan of a barcode on the medication container and/or identification of an order for the medication.

The pumping mechanism 16 (e.g., a peristaltic apparatus of the pump 10) starts (410). According to various implementations, the motor speeds of the pump 10 are pre-determined at their corresponding infusion flow rates. The pump software is programmed to adjust the speed faster or slower than the pre-determined speed if there is a mismatch between what the actual volume is infused at a given time versus the expected volume infused at a given time. As described previously with regard to FIG. 2, the disclosed vision system (e.g., image sensing device 20 and corresponding software) checks the fluid level within the container periodically, in real-time during the infusion (412). The software determines whether the fluid level reduction rate corresponds to the reduction expected at the set flow rate (414). In some implementations, the software determines whether the actual amount of fluid infused—as read by the image sensing device 20—differs from the estimated amount by at least a first threshold amount.

If the difference does not satisfy the first threshold amount, then the pump 10 continues the infusion as programmed (416). If it does then the software process continues by determining whether there is a severe disparity between the fluid level reading and an expected reading (418), for example, by determining whether a second threshold amount is satisfied (e.g., greater than the first threshold amount). The specifications of the acceptable difference between actual and expected volume infused may be set by the pump manufacturer, or the healthcare organization in which the pump is maintained. Such added security ensures that any gross inaccuracies are detected early and avoids potential harm to patients.

The second threshold amount may be for determining whether to dispatch an alarm. Thus, if the second threshold amount is satisfied, an alarm may sound at the pump (420) and, in some implementations, the pumping mechanism terminated, stopping the infusion (422).

If the second threshold amount is not satisfied then the pump software may adjust the motor speed of the pump to compensate (424). For example, if X mL is expected but the vision system is reporting X-0.02 mL then the pump motor may be increased to delivery the additional 0.02 mL volume within a predetermined time period. The predetermined time period may be selected for safe delivery of the particular medication being delivered (e.g., as indicated by way of step 402 or APR). The process continues until the infusion is completed (425).

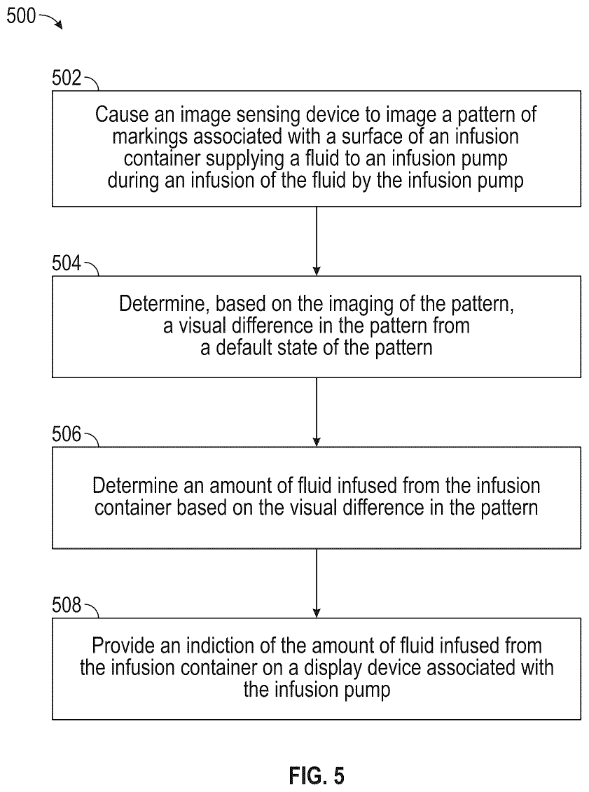

FIG. 5 depicts a second example process 500 for maintaining accuracy of an infusion pump using a vision-enabled fluid-flow detection system, according to various aspects of the subject technology. For explanatory purposes, the various blocks of example process 500 are described herein with reference to FIGS. 1-4, as well as the components and processes described herein. In some implementations, one or more of the blocks may be implemented apart from other blocks, and by one or more different processors or devices. Further, for explanatory purposes, the blocks of example process 500 are described as occurring in serial, or linearly. However, multiple blocks of example process 500 may occur in parallel. In addition, the blocks of example process 500 need not be performed in the order shown and one or more of the blocks of example process 500 need not be performed.

The subject technology provides a vision system for maintaining the accuracy of the infusion pump 10. As described previously, an image sensing device 20 is configured to image an infusion container 2 supplying a fluid to an infusion pump. A pattern of markings is associated with a surface of an infusion container and positioned such that the pattern is superimposed with a characteristic of a fluid within the infusion container when the infusion container is imaged by the image sensing device.

As described previously, the walls of the fluid container 2 may include a background pattern and a foreground pattern. The background pattern may be affixed to a first side of the infusion container and the foreground pattern is affixed to a second side of the infusion container, opposite the first side. When the infusion container is imaged by the vision system (e.g., by the image sensing device 20), the foreground pattern is in front of the fluid within the infusion container between the image sensing device and the infusion container, and the background pattern is behind the fluid in the infusion container. Because the infusion container is transparent, the image generated by the vision system includes the foreground pattern being visually modified by a combination of the background pattern, the characteristic of the fluid within the infusion container, and/or a current shape of the container. The portions of the modified foreground pattern are different based on a presence or absence of the fluid within the infusion container at the portions.

Accordingly, the image sensing device is caused to image the pattern of markings during an infusion of the fluid by the infusion pump (502). According to various implementations, the pattern of markings may be periodically monitored so that, as will be described further, a flow rate of the infusion pump 10 can be determined based on a difference in the pattern between a first and second time.

Based on the imaging of the pattern, the vision system determines a visual difference in the pattern from a default state of the pattern (504). According to various implementations, the vision system includes a statistical model or a machine learning model trained to recognize an amount of fluid remaining in the infusion container based on comparing the pattern to a training dataset of patterns and fluid characteristics. According to various implementations, the model may be trained with different backgrounds, colors of backgrounds, patterns, and a variety of ambient lighting. The vision system provides the image of the pattern to the model and receives, from the model in response, an amount of fluid remaining in the container 2. In some implementations, the vision system may captures the pattern iteratively. In this regard, the default state may be the pattern captured at a first time, which is then compared with the pattern captured at a second time.

The vision system determines an amount of fluid infused from the infusion container based on the visual difference in the pattern (506). Accordingly, in some implementations, the vision system may determine the amount of fluid infused from the container based on a difference between the amount of fluid remaining in the infusion container that was received from the model (e.g., a current iteration of the model prediction) and a prior amount of fluid determined to be in the infusion container.

In some implementations that use a deformable container such as an IV bag, determining the amount of fluid infused from the infusion container based on the visual difference in the pattern includes determining a change in size of the bag based on the pattern. For example, when the infusion is initiated, the clinician may input an identifier of the container 2. The input may be by way of direct input into the control interface of the pump or by scanning a barcode or QR code on the bag. The vision system then determines based on the identifier, one or more dimensions of the container and a default volume of the fluid within the container. These dimensions may include, for example, a deformable diameter that depends on a current volume of the fluid within the container. In some implementations, the identifier is used to associate the captured pattern in an image with the current size or diameter of the container so that the current volume of the fluid in the container can be calculated.

The vision system, for example, may query a database or lookup table in its associated memory system or in an external system based on the identifier. In some implementations, the query returns a correlation between a captured pattern image and a dimension. In some implementations, the dimensions are returned. The correlation and/or dimensions may be input to the model (e.g., statistical model or machine learning model) for the current infusion. In this regard, the deformable diameter may be determined based on the visual difference in the pattern, and the vision system may determine the current volume of fluid in the container based on the determined deformable diameter. Accordingly, the vision system may determine the amount of fluid infused from the infusion container based on a difference between the current volume and a previously determined volume.

In this regard, the vision system may periodically monitor the pattern with the image sensing device during the infusion, while at the same time periodically receiving, from a controller associated with the infusion pump, an estimated amount of fluid infused by the infusion pump over a predetermined period of time. The infusion pump controller receives information from an encoder wheel attached to the pumping motor. The number of encoder per revolution of the encoder wheel is known, as well as the motor speed for a given flow rate. Based on this information, the controller may calculate a volume infused for a given period of time. The vision system determines an actual amount of fluid infused by the infusion pump over the predetermined period of time based on the determined visual difference in the pattern. For example, the vision system may capture the pattern iteratively, and compare the pattern captured at a first time with the pattern captured at a second time. The software may then determine that the actual amount of fluid infused (as determined by the vision system) differs from the estimated amount. When the difference satisfies a threshold amount, the software may take action.

An indication of the amount of fluid infused from the infusion container is provided on a display device associated with the infusion pump (508). The indication may include, for example, an indication that the infusion pump is reporting an incorrect amount of fluid being infused, for example, when the difference between the amounts reported by the vision system and the pump's estimation system (e.g., based on the encoder) differ by more than the threshold amount. For example, the indication may be represented by an alert on a display screen integrated in the infusion pump 10 or associated with the infusion pump, such as in a display of computing device 22, a computing device associated with a clinician (e.g., a mobile device of the clinician or a computing device in a nurse's station for monitoring multiple infusion devices in a particular care area). The indication may be, for example, a flashing icon or a change in color of a flow or volume measurement. Additionally, or in the alternative, the indication may be or include an audible alarm.

In some implementations, on the vision system determining that the difference between the vision measurement and the controller's measurement (e.g., based on the encoder) reaches a threshold amount, the vision system may signal the pump to increase its motor speed to compensate for the difference; for example, reduce a variation between a flow rate calculated based on the estimated amount and a flow rate calculated based on the actual amount. In this regard, the infusion pump's infusion rate may be recalibrated to reflect the correct infusion rate. In some implementation, the vision system may signal the infusion pump to terminate the infusion.

According to various implementations, the vision system may utilize different thresholds for generating the alarm, increasing the motor speed, and/or terminating the infusion. For example, the alarm may be generated responsive to the fluid level reduction rate reaching a first threshold, the motor speed may be adjusted when a second threshold is reached, and the pump terminated when a third threshold is reached. In some implementations, these thresholds for adjusting the motor speed and generating the alarm may be the same. In some implementations, the threshold for the alarm may be lower than the threshold for adjusting the motor speed. In some implementations it may be greater. In some implementations, the threshold for terminating the pump may be greater than the threshold(s) for generating the alarm and/or adjusting the motor. In some implementations, the alarm is generated when the pump is terminated.

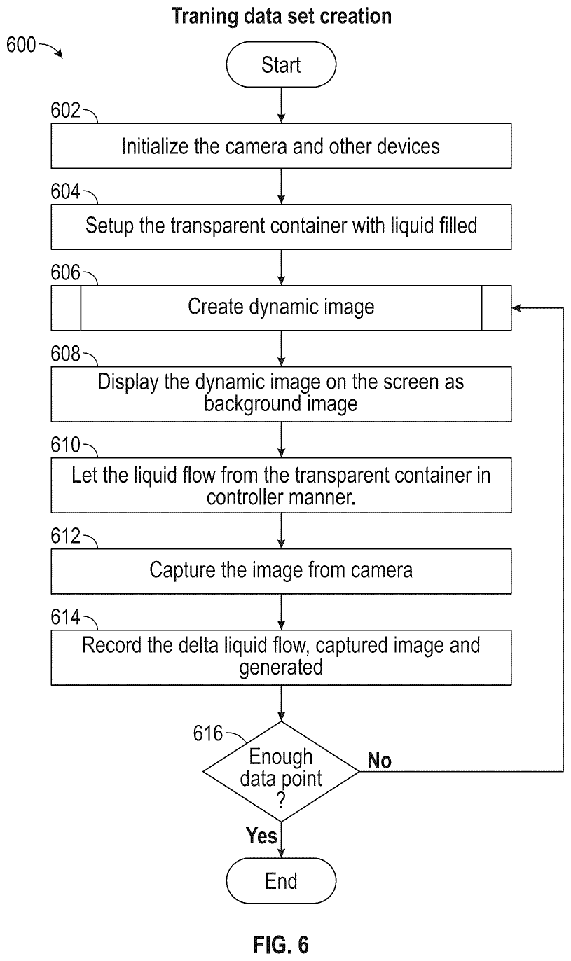

FIG. 6 depicts an example process for training the disclosed vision-enabled fluid-flow detection system to detect on or more patterns associated with a fluid container to detect fluid flow during a patient therapy, according to various aspects of the subject technology. For explanatory purposes, the various blocks of example process 600 are described herein with reference to FIGS. 1-5, as well as the components and processes described herein. In some implementations, one or more of the blocks may be implemented apart from other blocks, and by one or more different processors or devices. Further, for explanatory purposes, the blocks of example process 600 are described as occurring in serial, or linearly. However, multiple blocks of example process 600 may occur in parallel. In addition, the blocks of example process 600 need not be performed in the order shown and one or more of the blocks of example process 600 need not be performed.

According to the depicted example, the first step initializes the camera and other devices of the disclosed vision system (602). A fluid flow container is set up, including aligning the camera of the vision system with the container (604). The vision system images the container 2 and creates a dynamic image (606). The first image generated by the camera captures the previously described pattern in a default state. The dynamic image may optionally be displayed on a computer screen (or other screen associated with the pump) as a background image (608). The fluid is allowed to be expelled and/or drain from the container 2 in a controlled manner (610) and the vision system images the container 2 again to create a second dynamic image (612). The vision system records the delta fluid flow, captured image and data related to the generated image that was shown or printed on the container. The data, in some implementations, may include the specific image being displayed by the infusion device, as a background image 40 (see FIG. 3C) and within the field of view of the vision system. In some implementations, the data may include a hash code or other encoding or a reference identifier to indicate the image shown or printed. The vision system then determines whether there is enough data (e.g., from the image analysis and input to the statistical or machine learning model) to determine a new fluid level (614). If not, then the process 600 repeats steps 606 to 614.

When enough data is available to determine a fluid level (e.g., volume) then the process ends. The entire process 600 may be repeated multiple times for the container to ensure data accuracy. Each iteration may be associated with an identifier of the container used in the training so that the determined training data/model may be loaded by the vision system during runtime when the identifier is input into the system.

Many of the above-described example processes 500 and 600, and related programming and configuring features, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 7 is a conceptual diagram illustrating an example electronic system 700 for maintaining accuracy of an infusion pump using a vision-enabled fluid-flow detection system, according to aspects of the subject technology. Electronic system 700 may be a computing device for execution of software associated with one or more portions or steps of process 700, or components and processes provided by FIGS. 1-6, including but not limited to computing device 8, processor 514, computing hardware within an infusion device 10, or an operably connected remote device (e.g., a mobile device). Electronic system 700 may be representative, in combination with the disclosure regarding FIGS. 1-7. In this regard, electronic system 700 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 700 may include various types of computer readable media and interfaces for various other types of computer readable media. In the depicted example, electronic system 700 includes a bus 708, processing unit(s) 712, a system memory 704, a read-only memory (ROM) 710, a permanent storage device 702, an input device interface 714, an output device interface 706, and one or more network interfaces 716. In some implementations, electronic system 700 may include or be integrated with other computing devices or circuitry for operation of the various components and processes previously described.

Bus 708 collectively represents all system, peripheral, and chipset buses that communicatively connect the numerous internal devices of electronic system 700. For instance, bus 708 communicatively connects processing unit(s) 712 with ROM 710, system memory 704, and permanent storage device 702.

From these various memory units, processing unit(s) 712 retrieves instructions to execute and data to process, in order to execute the processes of the subject disclosure. The processing unit(s) can be a single processor or a multi-core processor in different implementations.

ROM 710 stores static data and instructions that are needed by processing unit(s) 712 and other modules of the electronic system. Permanent storage device 702, on the other hand, is a read-and-write memory device. This device is a non-volatile memory unit that stores instructions and data even when electronic system 700 is off. Some implementations of the subject disclosure use a mass-storage device (such as a magnetic or optical disk and its corresponding disk drive) as permanent storage device 702.

Other implementations use a removable storage device (such as a floppy disk, flash drive, and its corresponding disk drive) as permanent storage device 702. Like permanent storage device 702, system memory 704 is a read-and-write memory device. However, unlike storage device 702, system memory 704 is a volatile read-and-write memory, such as a random access memory. System memory 704 stores some of the instructions and data that the processor needs at runtime. In some implementations, the processes of the subject disclosure are stored in system memory 704, permanent storage device 702, and/or ROM 710. From these various memory units, processing unit(s) 712 retrieves instructions to execute and data to process in order to execute the processes of some implementations.

Bus 708 also connects to input and output device interfaces 714 and 706. Input device interface 714 enables the user to communicate information and select commands to the electronic system. Input devices used with input device interface 714 include, e.g., alphanumeric keyboards and pointing devices (also called “cursor control devices”). Output device interfaces 706 enables, e.g., the display of images generated by the electronic system 700. Output devices used with output device interface 706 include, e.g., printers and display devices, such as cathode ray tubes (CRT) or liquid crystal displays (LCD). Some implementations include devices such as a touchscreen that functions as both input and output devices.

Also, as shown in FIG. 7, bus 708 also couples electronic system 700 to a network (not shown) through network interfaces 716. Network interfaces 716 may include, e.g., a wireless access point (e.g., Bluetooth or WiFi) or radio circuitry for connecting to a wireless access point. Network interfaces 716 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 700 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.
Clause 1. A fluid flow detection system comprising: an image sensing device configured to image an infusion container supplying a fluid to an infusion pump; a pattern of markings associated with a surface of an infusion container and positioned such that the pattern is superimposed with a characteristic of a fluid within the infusion container when the infusion container is imaged by the image sensing device; and one or more processors configured to: cause the image sensing device to image the pattern of markings during an infusion of the fluid by the infusion pump; determine, based on the imaging of the pattern, a visual difference in the pattern from a default state of the pattern; determine a volume of fluid infused from the infusion container based on the visual difference in the pattern; determine a flow rate of infusion; and provide an indication of the volume of fluid infused from the infusion container on a display device associated with the infusion pump.
Clause 2. The fluid flow detection system of Clause 1, wherein determining the visual difference in the pattern comprises: providing the image of the pattern to a machine learning model trained to recognize a volume of fluid remaining in the infusion container based on comparing the pattern to a training dataset of patterns and fluid characteristics; and receiving, from the machine learning model responsive to providing the image to the machine learning model, the volume of fluid remaining in the infusion container, wherein the volume of fluid infused from the infusion container is based on a difference between the volume of fluid remaining in the infusion container that was received from the machine learning model and a prior volume of fluid determined to be in the infusion container.
Clause 3. The fluid flow detection system of Clause 1 or Clause 2, wherein the one or more processors is further configured to: determine that the infusion pump has initiated an infusion of the fluid; periodically monitor the pattern with the image sensing device during the infusion; and receive, from a controller associated with the infusion pump, an estimated volume of fluid infused by the infusion pump over a predetermined period of time; determine an actual volume of fluid infused by the infusion pump over the predetermined period of time based on the determined visual difference in the pattern; determine that the actual volume of fluid infused differs from the estimated volume by a threshold volume; and provide an indication that the infusion pump is reporting an incorrect volume of fluid being infused.
Clause 4. The fluid flow detection system of Clause 3, wherein the one or more processors is further configured to: image the default state of the pattern at a first time during the infusion; image the pattern at a second time during the infusion, wherein the second time is after the first time; and determine a flow rate of the infusion pump based on the visual difference in the pattern between the first time and the second time.
Clause 5. The fluid flow detection system of Clause 3, wherein the one or more processors is further configured to, responsive to determining that the actual volume of fluid infused differs from the estimated volume by a first threshold volume: adjust a motor speed of the infusion pump to reduce a variation between a flow rate calculated based on the estimated volume and a flow rate calculated based on the actual volume.
Clause 6. The fluid flow detection system of Clause 5, wherein the one or more processors is further configured to: signal the infusion pump to terminate the infusion when the actual volume of fluid infused differs from the estimated volume by a second threshold volume, wherein the infusion is terminated responsive to the signal.
Clause 7. The fluid flow detection system of any one of Clauses 1-6, wherein the one or more processors is further configured to: receive an identifier of the infusion container; determine, based on the identifier, one or more dimensions of the infusion container and a default volume of the fluid within the infusion container, wherein the one or more dimensions comprise a deformable diameter that depends on a current volume of the fluid within the container; determine the deformable diameter based on the visual difference in the pattern; and determine the current volume of fluid in the container based on the determined deformable diameter, wherein the volume of fluid infused from the infusion container is based on a difference between the current volume and a previously determined volume.
Clause 8. The fluid flow detection system of any one of Clauses 1-7, wherein the pattern of markings comprises: a background pattern and a foreground pattern wherein, when the infusion container is imaged by the image sensing device, the foreground pattern is in front of the fluid within the infusion container between the image sensing device and the infusion container, and the background pattern is behind the fluid in the infusion container, wherein the infusion container is transparent and the image generated by the image sensing device comprises the foreground pattern being visually modified by a combination of the background pattern, the characteristic of the fluid within the infusion container, and a current shape of the container, wherein portions of the modified foreground pattern are different based on a presence, volume of fluid or absence of the fluid within the infusion container at the portions.
Clause 9. The fluid flow detection system of Clause 8, wherein the background pattern is affixed to a first side of the infusion container and the foreground pattern is affixed to a second side of the infusion container, opposite the first side.
Clause 10. The fluid flow detection system of Clause 8, wherein the pattern of markings comprises a background pattern generated by a display device associated with the infusion pump, wherein the one or more processors is further configured to: generate the background pattern, wherein the display device is positioned behind the infusion container with respect to the image sensing device such that the image sensing device images the fluid within the container in front of the background pattern.
Clause 11. The fluid flow detection system of Clause 10, wherein the one or more processors is further configured to: determine one or more characteristics of the fluid within the infusion container; determine one or more parameters associated with the determined one or more characteristics, the one or more parameters being for setting a displayed visual characteristic of the background pattern; and adjusting the displayed visual characteristic of the background pattern.
Clause 12. The fluid flow detection system of Clause 11, wherein the one or more processors is further configured to: determine that at least one of the one or more parameters changed during the infusion; and dynamically adjust the displayed visual characteristic during the infusion.
Clause 13. A machine-implemented method, comprising: causing an image sensing device to image a pattern of markings associated with a surface of an infusion container supplying a fluid to an infusion pump during an infusion of the fluid by the infusion pump, the pattern of markings being positioned such that the pattern is superimposed with a characteristic of the fluid within the infusion container when the infusion container is imaged by the image sensing device; determining, based on the imaging of the pattern, a visual difference in the pattern from a default state of the pattern; determining an volume of fluid infused from the infusion container based on the visual difference in the pattern; and providing an indication of the volume of fluid infused from the infusion container on a display device associated with the infusion pump.
Clause 14. The machine-implemented method of Clause 13, wherein determining the visual difference in the pattern comprises: providing the image of the pattern to a model that is trained to recognize an volume of fluid remaining in the infusion container based on comparing the pattern to a training dataset of patterns and fluid characteristics, and receiving, from the model responsive to providing the image to the model, the volume of fluid remaining in the infusion container, wherein the volume of fluid infused from the infusion container is based on a difference between the volume of fluid remaining in the infusion container that was received from the model and a prior volume of fluid determined to be in the infusion container.
Clause 15. The machine-implemented method of Clause 13 or Clause 14, further comprising: determining that the infusion pump has initiated an infusion of the fluid; periodically monitoring the pattern with the image sensing device during the infusion; receiving, from a controller associated with the infusion pump, an estimated volume of fluid infused by the infusion pump over a predetermined period of time; determining an actual volume of fluid infused by the infusion pump over the predetermined period of time based on the determined visual difference in the pattern; determining that the actual volume of fluid infused differs from the estimated volume by a threshold volume; and responsive to determining that the actual volume of fluid infused differs from the estimated volume by a threshold volume, adjusting a motor speed of the infusion pump to reduce a variation between a flow rate calculated based on the estimated volume and a flow rate calculated based on the actual volume.
Clause 16. The machine-implemented method of any one of Clauses 13-15, further comprising: receiving an identifier of the infusion container; determining, based on the identifier, one or more dimensions of the container and a default volume of the fluid within the container, wherein the one or more dimensions comprise a deformable diameter that depends on a current volume of the fluid within the container; determining the deformable diameter based on the visual difference in the pattern; and determining the current volume of fluid in the container based on the determined deformable diameter, wherein the volume of fluid infused from the infusion container is based on a difference between the current volume and a previously determined volume.
Clause 17. The machine-implemented method of any one of Clauses 13-16, wherein the pattern of markings comprises: a background pattern and a foreground pattern wherein, when the infusion container is imaged by the image sensing device, the foreground pattern is in front of the fluid within the infusion container between the image sensing device and the infusion container, and the background pattern is behind the fluid in the infusion container, wherein the infusion container is transparent and the image generated by the image sensing device comprises the foreground pattern being visually modified by a combination of the background pattern, the characteristic of the fluid within the infusion container, and a current shape of the container, wherein portions of the modified foreground pattern are different based on a presence, volume of fluid present at that level of container or absence of the fluid within the infusion container at the portions.
Clause 18. The machine-implemented method of any one of Clauses 13-17, wherein the pattern of markings comprises a background pattern generated by a display device associated with the infusion pump, wherein the method further comprises: generating the background pattern, wherein the display device is positioned behind the infusion container with respect to the image sensing device such that the image sensing device images the fluid within the container in front of the background pattern.
Clause 19. The machine-implemented method of Clause 18, further comprising: determine one or more characteristics of the fluid within the infusion container; determine one or more parameters associated with the determined one or more characteristics, the one or more parameters being for setting a displayed visual characteristic of the background pattern; adjusting the displayed visual characteristic of the background pattern; determining that at least one of the one or more parameters changed during the infusion; and dynamically adjusting the displayed visual characteristic during the infusion.
Clause 20. A non-transitory machine-readable storage medium storing instructions thereon that, when executed by a machine, cause the machine to perform a method according to any one of claims 13-19.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration. ” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

What is claimed is:

1. A fluid flow detection system comprising:
an image sensing device configured to image an infusion container supplying a fluid to an infusion pump;
a pattern of markings associated with a surface of an infusion container and positioned such that the pattern is superimposed with a characteristic of a fluid within the infusion container when the infusion container is imaged by the image sensing device; and
one or more processors configured to:
cause the image sensing device to image the pattern of markings during an infusion of the fluid by the infusion pump;
determine, based on the imaging of the pattern, a visual difference in the pattern from a default state of the pattern;
determine a volume of fluid infused from the infusion container based on the visual difference in the pattern;
determine a flow rate of infusion; and
provide an indication of the volume of fluid infused from the infusion container on a display device associated with the infusion pump.

2. The fluid flow detection system of claim 1, wherein determining the visual difference in the pattern comprises:
providing the image of the pattern to a machine learning model trained to recognize a volume of fluid remaining in the infusion container based on comparing the pattern to a training dataset of patterns and fluid characteristics; and
receiving, from the machine learning model responsive to providing the image to the machine learning model, the volume of fluid remaining in the infusion container,
wherein the volume of fluid infused from the infusion container is based on a difference between the volume of fluid remaining in the infusion container that was received from the machine learning model and a prior volume of fluid determined to be in the infusion container.

3. The fluid flow detection system of claim 1, wherein the one or more processors is further configured to:
determine that the infusion pump has initiated an infusion of the fluid;
periodically monitor the pattern with the image sensing device during the infusion; and
receive, from a controller associated with the infusion pump, an estimated volume of fluid infused by the infusion pump over a predetermined period of time;
determine an actual volume of fluid infused by the infusion pump over the predetermined period of time based on the determined visual difference in the pattern;
determine that the actual volume of fluid infused differs from the estimated volume by a threshold volume; and
provide an indication that the infusion pump is reporting an incorrect volume of fluid being infused.

4. The fluid flow detection system of claim 3, wherein the one or more processors is further configured to:
image the default state of the pattern at a first time during the infusion;
image the pattern at a second time during the infusion, wherein the second time is after the first time; and
determine a flow rate of the infusion pump based on the visual difference in the pattern between the first time and the second time.

5. The fluid flow detection system of claim 3, wherein the one or more processors is further configured to, responsive to determining that the actual volume of fluid infused differs from the estimated volume by a first threshold volume:
adjust a motor speed of the infusion pump to reduce a variation between a flow rate calculated based on the estimated volume and a flow rate calculated based on the actual volume.

6. The fluid flow detection system of claim 5, wherein the one or more processors is further configured to:
signal the infusion pump to terminate the infusion when the actual volume of fluid infused differs from the estimated volume by a second threshold volume,
wherein the infusion is terminated responsive to the signal.

7. The fluid flow detection system of claim 1, wherein the one or more processors is further configured to:
receive an identifier of the infusion container;
determine, based on the identifier, one or more dimensions of the infusion container and a default volume of the fluid within the infusion container, wherein the one or more dimensions comprise a deformable diameter that depends on a current volume of the fluid within the container;
determine the deformable diameter based on the visual difference in the pattern; and
determine the current volume of fluid in the container based on the determined deformable diameter, wherein the volume of fluid infused from the infusion container is based on a difference between the current volume and a previously determined volume.

8. The fluid flow detection system of claim 1, wherein the pattern of markings comprises:
a background pattern and a foreground pattern wherein, when the infusion container is imaged by the image sensing device, the foreground pattern is in front of the fluid within the infusion container between the image sensing device and the infusion container, and the background pattern is behind the fluid in the infusion container, wherein the infusion container is transparent and the image generated by the image sensing device comprises the foreground pattern being visually modified by a combination of the background pattern, the characteristic of the fluid within the infusion container, and a current shape of the container, wherein portions of the modified foreground pattern are different based on a presence, volume of fluid or absence of the fluid within the infusion container at the portions.

9. The fluid flow detection system of claim 8, wherein the background pattern is affixed to a first side of the infusion container and the foreground pattern is affixed to a second side of the infusion container, opposite the first side.

10. The fluid flow detection system of claim 8, wherein the pattern of markings comprises a background pattern generated by a display device associated with the infusion pump, wherein the one or more processors is further configured to:
generate the background pattern, wherein the display device is positioned behind the infusion container with respect to the image sensing device such that the image sensing device images the fluid within the container in front of the background pattern.

11. The fluid flow detection system of claim 10, wherein the one or more processors is further configured to:
determine one or more characteristics of the fluid within the infusion container;
determine one or more parameters associated with the determined one or more characteristics, the one or more parameters being for setting a displayed visual characteristic of the background pattern; and
adjusting the displayed visual characteristic of the background pattern.

12. The fluid flow detection system of claim 11, wherein the one or more processors is further configured to:
determine that at least one of the one or more parameters changed during the infusion; and
dynamically adjust the displayed visual characteristic during the infusion.

13. A machine-implemented method, comprising:
causing an image sensing device to image a pattern of markings associated with a surface of an infusion container supplying a fluid to an infusion pump during an infusion of the fluid by the infusion pump, the pattern of markings being positioned such that the pattern is superimposed with a characteristic of the fluid within the infusion container when the infusion container is imaged by the image sensing device;
determining, based on the imaging of the pattern, a visual difference in the pattern from a default state of the pattern;
determining an volume of fluid infused from the infusion container based on the visual difference in the pattern; and
providing an indication of the volume of fluid infused from the infusion container on a display device associated with the infusion pump.

14. The machine-implemented method of claim 13, wherein determining the visual difference in the pattern comprises:
providing the image of the pattern to a model that is trained to recognize an volume of fluid remaining in the infusion container based on comparing the pattern to a training dataset of patterns and fluid characteristics, and
receiving, from the model responsive to providing the image to the model, the volume of fluid remaining in the infusion container,
wherein the volume of fluid infused from the infusion container is based on a difference between the volume of fluid remaining in the infusion container that was received from the model and a prior volume of fluid determined to be in the infusion container.

15. The machine-implemented method of claim 13, further comprising:
determining that the infusion pump has initiated an infusion of the fluid;
periodically monitoring the pattern with the image sensing device during the infusion;
receiving, from a controller associated with the infusion pump, an estimated volume of fluid infused by the infusion pump over a predetermined period of time;
determining an actual volume of fluid infused by the infusion pump over the predetermined period of time based on the determined visual difference in the pattern;
determining that the actual volume of fluid infused differs from the estimated volume by a threshold volume; and
responsive to determining that the actual volume of fluid infused differs from the estimated volume by a threshold volume, adjusting a motor speed of the infusion pump to reduce a variation between a flow rate calculated based on the estimated volume and a flow rate calculated based on the actual volume.

16. The machine-implemented method of claim 13, further comprising:
receiving an identifier of the infusion container;
determining, based on the identifier, one or more dimensions of the container and a default volume of the fluid within the container, wherein the one or more dimensions comprise a deformable diameter that depends on a current volume of the fluid within the container;
determining the deformable diameter based on the visual difference in the pattern; and
determining the current volume of fluid in the container based on the determined deformable diameter, wherein the volume of fluid infused from the infusion container is based on a difference between the current volume and a previously determined volume.

17. The machine-implemented method of claim 13, wherein the pattern of markings comprises:
a background pattern and a foreground pattern wherein, when the infusion container is imaged by the image sensing device, the foreground pattern is in front of the fluid within the infusion container between the image sensing device and the infusion container, and the background pattern is behind the fluid in the infusion container, wherein the infusion container is transparent and the image generated by the image sensing device comprises the foreground pattern being visually modified by a combination of the background pattern, the characteristic of the fluid within the infusion container, and a current shape of the container, wherein portions of the modified foreground pattern are different based on a presence, volume of fluid present at that level of container or absence of the fluid within the infusion container at the portions.

18. The machine-implemented method of claim 13, wherein the pattern of markings comprises a background pattern generated by a display device associated with the infusion pump, wherein the method further comprises:
generating the background pattern, wherein the display device is positioned behind the infusion container with respect to the image sensing device such that the image sensing device images the fluid within the container in front of the background pattern.

19. The machine-implemented method of claim 18, further comprising:
determine one or more characteristics of the fluid within the infusion container;
determine one or more parameters associated with the determined one or more characteristics, the one or more parameters being for setting a displayed visual characteristic of the background pattern;
adjusting the displayed visual characteristic of the background pattern;
determining that at least one of the one or more parameters changed during the infusion; and
dynamically adjusting the displayed visual characteristic during the infusion.

20. A non-transitory machine-readable storage medium storing instructions thereon that, when executed by a machine, cause the machine to perform a method according to claim 13.
